(19) United States
(12) Patent Application Publication
(10) Pub. No.: US 2009/0177157 A1
(43) Pub. Date: Jul. 9, 2009

(54) MICROPUMP WITH AT LEAST ONE GAS RELEASING MATERIAL

(75) Inventors: Michel Paul Barbara Van Bruggen, Helmond (NL); Hendrika Cecilia Krijnsen, Boxtel (NL); Geert Langereis, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, Eindhoven (NL)

(21) Appl. No.: 12/298,812

(22) PCT Filed: Apr. 18, 2007
(86) PCT No.: PCT/IB07/51392

§ 371 Date: Oct. 28, 2008

(30) Foreign Application Priority Data
Apr. 28, 2006 (EP) 06113249.4

(51) Int. Cl. A61M 5/00

(52) U.S. Cl. 604/143

(57) ABSTRACT

The invention relates to a micropump comprising at least one gas releasing material which drives a piston towards a second material containing a drug material which is released through a semi permeable membrane and/or flow restrictor

The present invention is directed to the field of micropumps.

Micropumps for the controlled delivery of drugs, e.g. inside a patient, are widely known in the field. E.g. WO 2005/032524, which is considered to be incorporated herein by reference, discloses a micropump capable of delivering a drug with an ascending release profile, which is caused by osmotic pressure.

However, known micropumps have the disadvantage that usually the osmotic pressure used to control the release of the drugs cannot be controlled in a sufficient and easy manner. E.g. in the micropump as disclosed in WO 2005/032524, there is only the possibility to increase the delivery rate over time, whereas a decrease is impossible. Furthermore, this control is pre-programmed and cannot be changed on demand, nor by means of a remote control.

A further micropump is disclosed in Bohm et al., Journal of Biomedical Microdevices, 1999, 121-130, which is considered to be incorporated herein by reference. In this microdevice, electrolysis of water is used to move a fluid through microchannels.

However, in this microdevice the flow rate is extremely temperature sensitive and depends on the temperature change as well as on the total amount of gas present in the electrolysis chamber.

### SUMMARY OF THE INVENTION

It is an object of the present invention to provide a micropump in which the pressure can be accurately controlled and in which the temperature sensitivity is significantly decreased.

This object is achieved by a micropump according to claim 1 of the present invention. Accordingly, a micropump is provided comprising at least one first reservoir and at least one second reservoir in which the drug to be released by the micropump is located, and at least one movable piston and/or deformable membrane is provided between the first reservoir and the second reservoir, wherein at least one gas releasing material is provided within the first reservoir and the second reservoir comprises a semi permeable material and/or a flow restrictor through which the drug is released by the micropump.

By doing so, for most applications at least one of the following advantages can be achieved:
The flow of drugs induced by the micropump is less sensitive to small changes in temperature and pressure and can be controlled reliably
The required energy is only small and is directly proportional to the amount of gas formed by the gas releasing material
The required electric potential will be only in the order of a few Volts

A flow restrictor in the sense of the present invention means and/or includes a thin channel-shaped outlet from the second reservoir towards the outside.

According to an embodiment of the present invention, the permeability of the semi permeable material is ≧10⁻²⁶ m² and ≦10⁻¹⁴ m². In most applications within the present invention, this has increased the accuracy and effectiveness of the drug flow induced by the micropump.

According to an embodiment of the present invention, the permeability of the first semi permeable material is ≧10⁻²⁴ m² and ≦10⁻¹⁶ m².

According to an embodiment of the present invention, the permeability of the first semi permeable material is ≧10⁻²³ m² and ≦10⁻¹⁷ m².

According to an embodiment of the present invention, the surface area of the part of the semi permeable material that projects towards the at least second reservoir is ≧10⁻⁷ m² and ≦10⁻⁴ m².

According to an embodiment of the present invention, the surface area of the part of the semi permeable material that projects towards the at least second reservoir is ≧5*10⁻⁶ m² and ≦8*10⁻⁶ m².

According to an embodiment of the present invention, the surface area of the part of the semi permeable material that projects towards the at least second reservoir is ≧10⁻⁶ m² and ≦5*10⁻⁶ m².

According to an embodiment of the present invention, the at least one gas releasing material releases at least one gas selected from the group comprising CO₂, N₂, O₂, H₂, NH₃, CH₄ during operation of the micropump.

According to an embodiment of the present invention, the at least one gas releasing material is selected from the groups comprising water, peroxides, perchlorides, chlorides, chlorates, carbonates, formaldehyde, aldehydes, formic acid, acetic acid, carboxylic acids, alcohols, nitrates, ammonia and mixtures thereof.

According to an embodiment of the present invention, the product of permeability and surface area of the semi permeable membrane is ≧10⁻²⁶ m⁴ and ≧10⁻²⁰ m⁴.

According to an embodiment of the present invention, the product of permeability and surface area of the semi permeable membrane is ≧10⁻²⁵ m⁴ and ≦10⁻²¹ m⁴.

According to an embodiment of the present invention, the product of permeability and surface area of the semi permeable membrane is ≧10⁻²⁴ m⁴ and ≦10⁻²² m⁴.

According to an embodiment of the present invention, the longitudinal thickness of the semi permeable material is ≧10⁻⁵ m and ≦10⁻¹ m.

According to an embodiment of the present invention, the longitudinal thickness of the semi permeable material is ≧5*10⁻⁴ m and ≦10⁻² m.

According to an embodiment of the present invention, the longitudinal thickness of the semi permeable material is ≧7.5*10⁻⁴ m and ≦5*10⁻³ m.

According to an embodiment of the present invention, the quotient of the permeability of the semi permeable membrane and the longitudinal thickness of the semi permeable material is ≧10⁻¹⁹ m and ≦10⁻¹¹ m.

According to an embodiment of the present invention, the quotient of the permeability of the semi permeable membrane and the longitudinal thickness of the semi permeable material is ≧10⁻¹⁷ m and ≦10⁻¹² m.

According to an embodiment of the present invention, the quotient of the permeability of the semi permeable membrane and the longitudinal thickness of the semi permeable material is ≧10⁻¹⁵ m and ≦5*10⁻¹³ m.

According to an embodiment of the present invention, the length L of the flow restrictor is ≧0.01 cm and ≦10 cm, preferably ≧0.1 cm and ≦5 cm. It should be noted that the design of the flow restrictor may be straight, however, the flow restrictor may have any form such as curved or spirally wound.

According to an embodiment of the present invention, the diameter of the flow restrictor is ≧10 μm and ≦500 μm, preferably ≧50 μm and ≦250 μm.

According to an embodiment of the present invention, the ratio of the length L to the diameter d of the flow restrictor is ≧20:1 and ≦5,000:1, preferably ≧200:1 and ≦1,000:1

According to an embodiment of the present invention, the micropump comprises at least two second reservoirs and at least two movable pistons, and the first reservoir is divided into at least two electrolysis chambers. In a number of applications, this has been shown to be a suitable embodiment within the present invention, especially when gas releasing compounds are used which release two gases, e.g. during electrolysis at the anode and the cathode.

According to an embodiment of the present invention, the micropump comprises at least one sensing and/or controlling means for controlling at least one of the following features:
the position of the piston and/or the deformable membrane
the amount of gas released by the gas releasing material
the temperature inside the first and/or second chamber(s), the drug and/or the gas.

In most applications within the present invention, this allows a more reliable control of the micropump.

According to an embodiment of the present invention, the at least two electrolysis chambers within the first reservoir are linked via a porous plug. This allows conduction between the two electrolysis chambers but prevents gas from going from one electrolysis chamber to the other electrolysis chamber.

The invention furthermore relates to a method of releasing a drug from a micropump as described above, wherein osmotic pressure is induced by charging the at least one chargeable material through the use of electric current.

A micropump according to the present invention may be used in a broad variety of systems and/or applications, including amongst them one or more of the following:
drug delivery systems
liquid absorbers
sample handling devices
micro valves
analytical devices

The aforementioned components, as well as the claimed components and the components to be used in accordance with the invention in the described embodiments, are not subject to any special exceptions with respect to their size, shape, material selection and technical concept, so that the selection criteria known in the pertinent field can be applied without limitations.

Additional details, characteristics and advantages of the object of the invention are disclosed in the subclaims, the Figures and the following description of the respective Figures and examples, which—in exemplary fashion—show several micropumps according to three embodiments of the present invention.

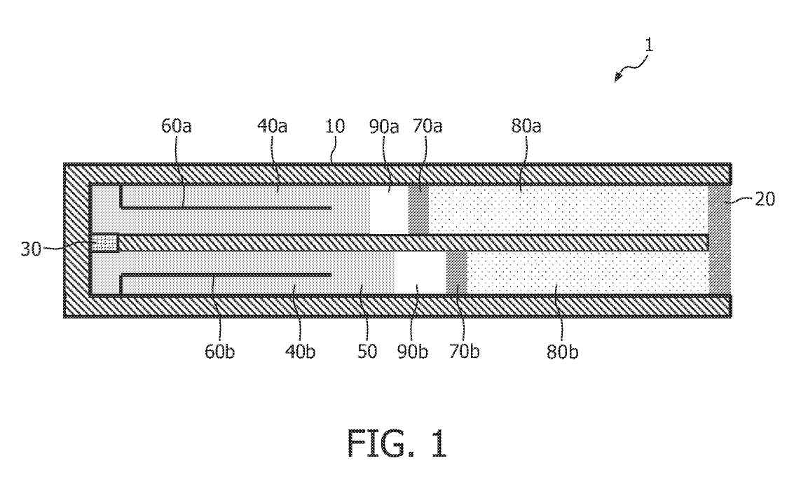

FIG. 1 shows a very schematic longitudinal cut-out view of a micropump according to one embodiment of the present invention;

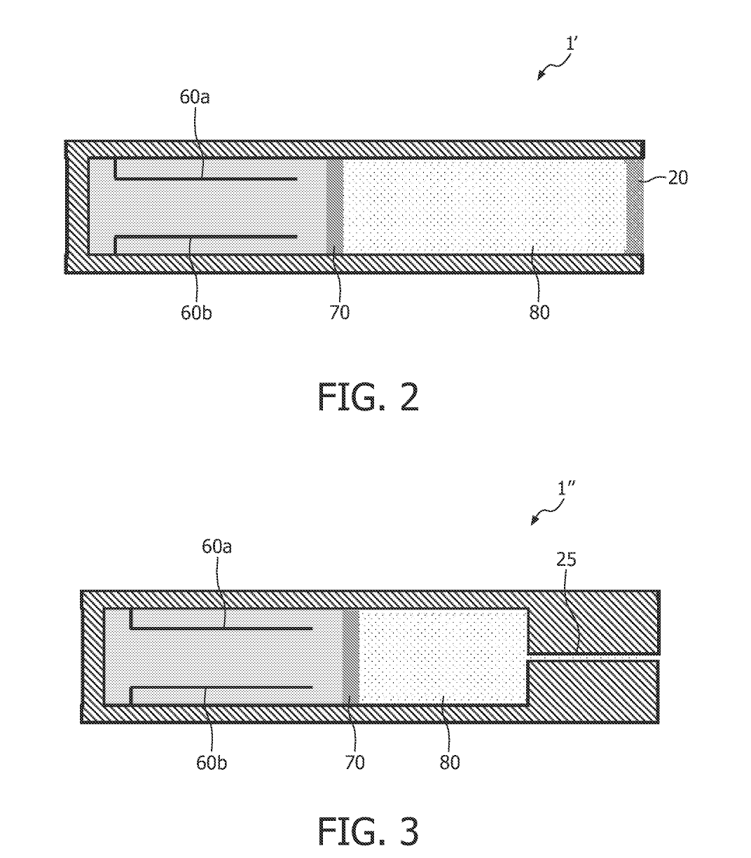

FIG. 2 shows a very schematic longitudinal cut-out view of a micropump according to a second embodiment of the present invention; and

FIG. 3 shows a very schematic longitudinal cut-out view of a micropump according to a third embodiment of the present invention;

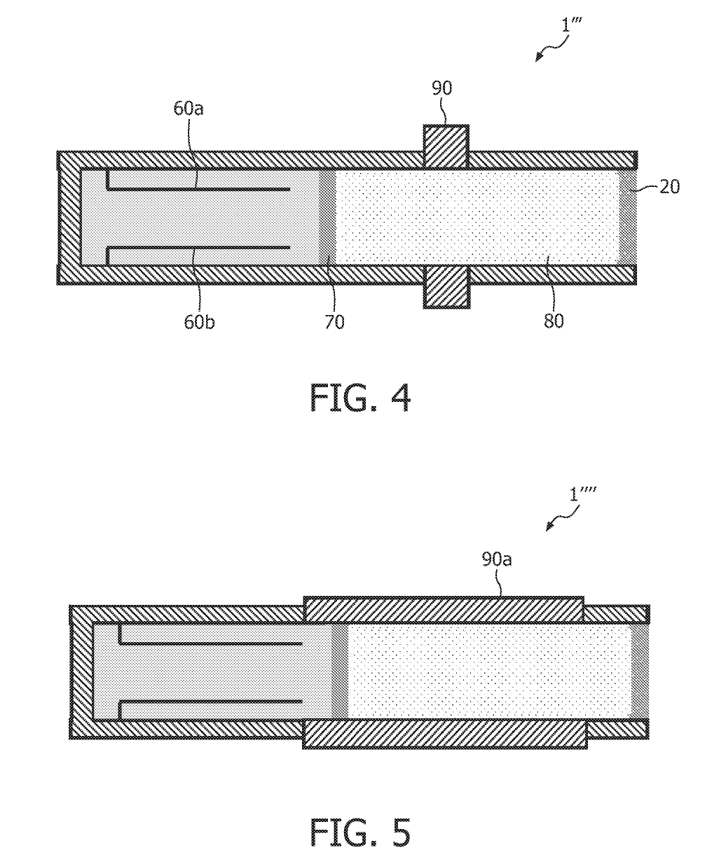

FIG. 4 shows a very schematic longitudinal cut-out view of a micropump according to a fourth embodiment of the present invention, including a temperature- and pressure-sensing means; and

FIG. 5 shows a very schematic longitudinal cut-out view of a micropump according to a fourth embodiment of the present invention, including a piston-positioning sensing means.

FIG. 1 shows a very schematic longitudinal cut-out view of a micropump 1 according to one embodiment of the present invention. The pump 1 comprises a pump body 10, which, seen in cross-section (not shown in the Figs.), may be circular, elliptical, square or rectangular. At one end of the pump body there is provided a semi permeable membrane 20.

The pump body 10 divides the first chamber into two electrolysis chambers 40a and 40b, which are each terminated at one end by a movable piston 70a, 70b, respectively. The electrolysis chambers 40a and 40b are linked via a porous plug.

On the other side of the pistons 70a, 70b, there are two second chambers 80a, 80b, respectively, which contain the drug to be delivered to the outside through the membrane 20.

Inside the first chamber there is one gas releasing material 50, which in this embodiment is simply water.

Upon electrolysis at the electrodes 60a and 60b, gas 90a and 90b (which is oxygen and hydrogen, depending on which electrode is the anode or the cathode) is released, moving the pistons 70a and 70b towards the right side, thereby causing the drug to be released.

It should be noted that an increase in temperature will first lead to an increase in pressure of the gases 90a and 90b without release of drug solution from the second chambers 80a, 80b, since the piston/membrane “arrangement” will have a certain “inertia” or “hydrodynamic resistance”. The temperature-induced pressure increase in the electrolysis chamber will therefore decrease only slowly and thus the temperature-induced increase of the flow rate will only be small, allowing the device to make adjustments to the drug administration protocol such that significant overdosing is circumvented

The drug could be sufentanil, fentanil, morphine, leuprolide acetate, insulin, psychotropics, contraceptive agents, growth hormones or other proteins, peptides, enzymes, genes, factors and hormones.

FIG. 2 shows a very schematic longitudinal cut-out view of a micropump 1′ according to a second embodiment of the present invention. This embodiment differs from that of FIG. 1 merely in that only one gas is released by the gas releasing material; as a result, a division into two different first and second chambers is not needed.

FIG. 3 shows a very schematic longitudinal cut-out view of a micropump 1″ according to a second embodiment of the present invention. This embodiment differs from that of FIGS. 1 and 2 in that a flow restrictor 25 is used instead of a semi permeable membrane as in the first and second embodiment. The flow restrictor 25 is drawn very schematically for visibility reasons. In most applications within the present invention, especially the diameter will most likely be much smaller than described above.

FIG. 4 shows a very schematic longitudinal cut-out view of a micropump 1′″ according to a fourth embodiment of the present invention, including a temperature- and/or pressure-sensing means. This temperature sensing means is, in this embodiment, provided in the form of a ring-shaped temperature and/or pressure sensor 90 which measures the temperature and/or pressure in the second chamber. This allows taking into account temperature and/or pressure changes within the micropump which might otherwise lead to unwanted drug release.

FIG. 5 shows a very schematic longitudinal cut-out view of a micropump 1′″ according to a fourth embodiment of the present invention, including a piston-position sensing means. This sensing means 90a is provided in the form of a longitudinal cylinder. The actual positioning of the piston can be measured via known techniques, e.g. in that the piston comprises a material which causes inductive changes inside the sensing means 90a, or any other known technique in the field. Such a sensing means allows for many applications to improve the control of the micropump. The particular combinations of elements and features in the above detailed embodiments are exemplary only; the interchanging and substitution of these teachings with other teachings in this document and in the patents/applications incorporated herein by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended to be construed in a limiting sense. The invention's scope is defined in the following claims and the equivalents thereto. Furthermore, reference signs used in the description and claims do not limit the scope of the invention as claimed.

1. A micropump for drug delivery comprising at least one first reservoir and at least one second reservoir in which the drug to be released by the micropump is located, and at least one movable piston and/or deformable membrane is provided between the first reservoir and the second reservoir, wherein at least one gas releasing material is provided within the first reservoir and the second reservoir comprises a semi permeable material and/or flow restrictor through which the drug is released by the micropump.

2. The micropump of claim 1, wherein the at least one gas releasing material releases at least one gas selected from the group comprising CO₂, N₂, O₂, H₂, NH₃, CH₄ during operation of the micropump.

3. The micropump of claim 1, wherein the at least one gas releasing material is selected from the groups comprising water, peroxides, perchlorides, chlorides, chlorates, carbonates, formaldehyde, aldehydes, formic acid, acetic acid, carboxylic acids, alcohols, nitrates, ammonia and mixtures thereof.

4. The micropump of claim 1, wherein the permeability of the semi permeable material is ≧10-26 m2 and ≦10-14 m2.

5. The micropump of claim 1, wherein the surface area of the part of the semi permeable material that projects towards the at least second reservoir is ≧10-7 m2 and ≦10-5 m2.

6. The micropump of claim 1, wherein the quotient of the permeability of the semi permeable membrane and the longitudinal thickness of the semipermeable material is ≧10-15 m and ≦10-11 m.

7. The micropump of claim 1, wherein the length L of the flow restrictor is ≧0.01 cm and ≦10 cm, preferably ≧0.1 cm and ≦5 cm.

8. The micropump of claim 1, wherein the diameter of the flow restrictor is ≧10 μm and ≦500 μm.

9. A method of releasing a drug from a micropump of claim 1, wherein gas is released by the gas releasing material to move the piston.

10. A system comprising a micropump according to claim 1, the system having one or more applications selected from the group consisting of:
drug delivery systems
liquid absorbers
sample handling devices
micro valves
